(19) United States
(12) Patent Application Publication
(10) Pub. No.: US 2010/0071695 A1
(43) Pub. Date: Mar. 25, 2010

(54) PATIENT WYE WITH FLOW TRANSDUCER

(76) Inventor: Ron Thiessen, Maple Ridge (CA)

Correspondence Address: NELLCOR PURITAN BENNETT LLC;ATTN: IP LEGAL, 6135 Gunbarrel Avenue, Boulder, CO, 80301, US

(21) Appl. No.: 12/235,908

(22) Filed: Sep. 23, 2008

(51) Int. Cl. A61M 16/00

(52) U.S. Cl. 128/204.18

(57) ABSTRACT

A patient wye for use in connection with a ventilator having one or more integrated transducers is provided. A transducer is integrated into the patient wye by locating the transducer within a branch of the patient wye. A transducer can be located between the center point of the wye and the port of the branch with which it is associated. More particularly, the transducer can be located so that it is on a side of a wall defining the branch in which it is placed that is opposite a connection surface for attaching tubing associated with a corresponding limb of the ventilator to the patient wye. Accordingly, flow or other transducers can be added to a patient circuit without necessarily increasing the volume of the patient circuit.

### FIELD

The present invention is generally directed to a patient wye for use in connection with a mechanical ventilator. More particularly, the present invention is directed to a patient wye having one or more integrated flow transducers.

### BACKGROUND

Ventilators are used to provide a breathing gas to a patient who is unable to breathe without assistance. In modern medical facilities, pressurized air and oxygen sources are often available from wall outlets. Accordingly, ventilators may provide pressure regulating valves connected to centralized sources of pressurized air and pressurized oxygen. The pressure regulating valves function to regulate flow so that respiratory air having a desired concentration of oxygen is supplied to the patient at desired pressures and rates. Ventilators capable of operating independently of external sources of pressurized air and oxygen are also available.

While operating a ventilator, it is desirable to monitor the rate at which breathing gas is supplied to the patient. Accordingly, some systems have interposed flow or pressure transducers between the patient supply tube, and the patient wye, which typically connects the inspiratory and expiratory limbs of the patient circuit to the patient supply tube. Because the inclusion of a transducer between the patient wye and the patient increases the volume or dead space of the patient side of the wye, the problem of rebreathing by the patient of exhaled gas is increased. In particular, when the patient exhales, the patient supply volume can remain full of carbon dioxide that is not flushed out. Accordingly, when the next breath occurs, that volume of carbon dioxide is delivered back to the patient.

Some ventilator systems monitor flows within the inspiratory and expiratory sides of the patient circuit using transducers coupled to the circuit, but positioned within the ventilator itself. However, some practitioners would prefer that monitoring occur as close as possible to the patient in some applications. Although transducers can be added to the patient circuit, the accompanying increase in the length and volume of the flow paths can be undesirable.

### SUMMARY

A patient wye for placing the inspiratory, expiratory and patient limbs of a patient ventilator system in communication with one another is provided that incorporates at least one integrated flow transducer. In particular, a flow transducer is located between the center point of the patient wye and the port of the wye for the section or limb of the patient circuit being monitored. In accordance with at least some embodiments of the present invention, a flow transducer integrated with a patient wye can be located on a side of the conduit forming a branch of the wye opposite a connection surface for a tube or other component connecting to the patient wye at the port associated with the transducer and with that branch.

In accordance with embodiments of the present invention, transducers integrated with the patient wye can be supplied for one or more branches of the patient wye, in any combination. For example, a transducer may be associated with each of the inspiratory and expiratory branches of the patient wye, or any other pair of branches. As another example, the patient wye may incorporate a single transducer that is associated with the patient branch of the patient wye. As still another example, a flow transducer may be associated with all three branches of the patient wye.

Additional features and advantages of embodiments of the present invention will become more readily apparent from the following description, particularly when taken together with the accompanying drawings.

### BRIEF DESCRIPTION OF THE DRAWINGS

FIG. 1 is a depiction of a mechanical ventilation system in accordance with embodiments of the present invention;

FIGS. 2A-2E depict example configurations of a patient wye in accordance with embodiments of the present invention;

FIGS. 3A-3B illustrate different flow transducer arrangements in accordance with embodiments of the present invention;

FIG. 4 is a cross section of a flow transducer that can be integrated with and mounted in a patient wye in accordance with embodiments of the present invention;

FIG. 5 is a flow chart depicting aspects of a process for operating a patient wye with one or more integrated flow transducers in accordance with embodiments of the present invention; and

FIG. 6 is a flow chart depicting aspects of a process for calibration and leak detection in a patient wye with one or more integrated flow transducers in accordance with embodiments of the present invention.

### DETAILED DESCRIPTION

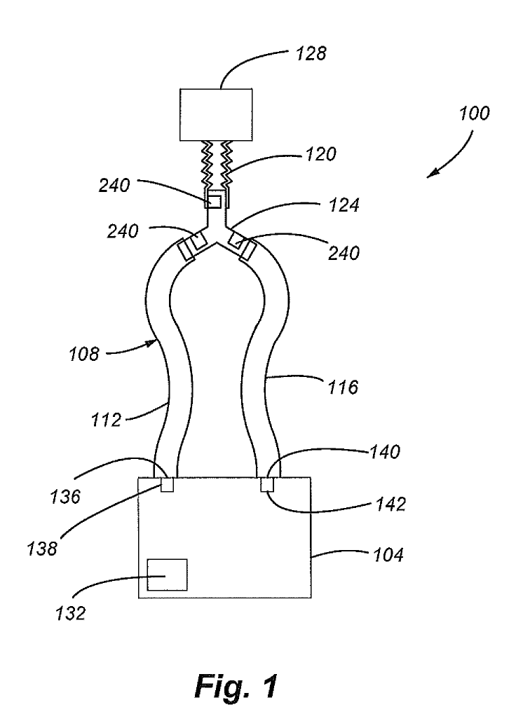

FIG. 1 is a depiction of a mechanical ventilation system 100 in accordance with embodiments of the present invention. In general, the system 100 includes a ventilator 104 connected to a patient circuit 108. The patient circuit includes an inspiratory limb 112, an expiratory limb 116 and a patient limb 120. The inspiratory 112, expiratory 116 and patient 120 limbs are interconnected to one another by a patient wye 124. The inspiratory limb 112 and expiratory limb 116 limb connect the patient wye 124 to a supply port 136 and a return 140 port, respectively, provided by the ventilator 104. The patient supply 120 interconnects the patient wye 124 to a patient breathing apparatus 128. Examples of a patient breathing apparatus include a breathing mask, a tracheostomy tube, an endotracheal tube, and abreathing mouthpiece

The ventilator 104 generally operates to provide a breathing gas to the inspiratory limb 112 for delivery to the patient breathing apparatus 128 via the patient wye 124 and the patient supply branch 120. The ventilator 104 may receive exhaled air from the patient that is delivered to an exhalation valve or similar device on the ventilator via the patient breathing apparatus 128, the patient supply limb 120, the patient wye 124, and the expiratory limb 116. The ventilator 104 may include supply sensors 138 and return sensors 142 flow. Sensors 138, 142 may be flow and/or pressure transducers to monitor the flow and/or pressure of supplied and returned gas, respectively. In accordance with embodiments of the present invention, information regarding the flow of gas through one or more of the limbs 112, 116, 120 of the patient circuit 108 is provided to a controller 132 included in or associated with the ventilator 104 by one or more transducers 240 integrated with the patient wye 124.

In general the patient wye 124 is an open, three port connector that places the patient limb 120 of the patient circuit 108 in communication with the inspiratory limb 112 and the expiratory limb 116 of the patient circuit 108.

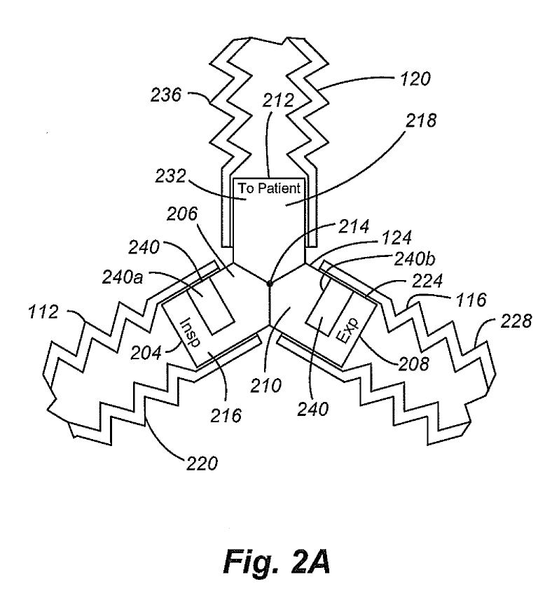

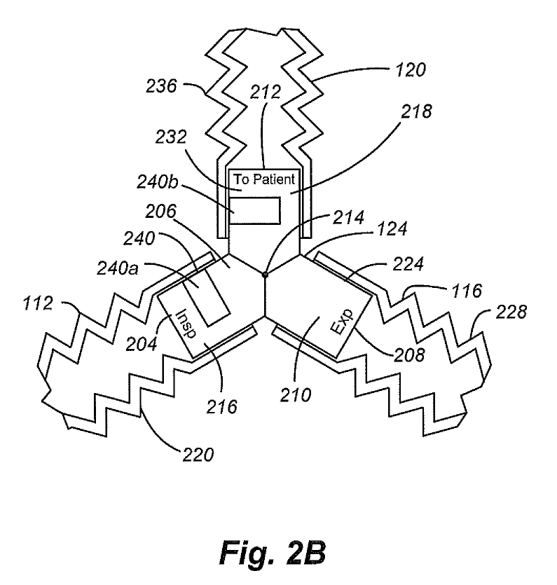

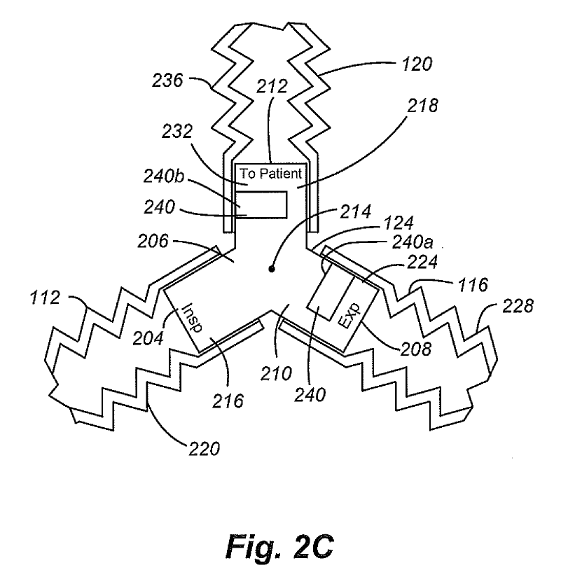

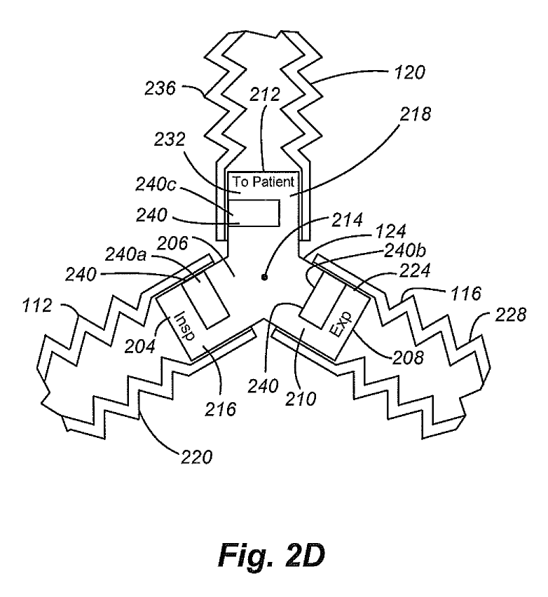

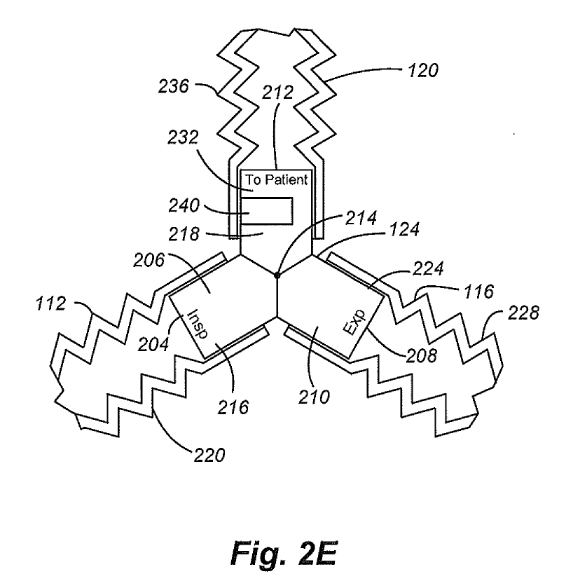

FIGS. 2A-2E illustrate different configurations of a patient wye 124 in accordance with embodiments of the present invention. As such, the patient wye 124 includes an inspiratory port 204, an expiratory port 208 and a patient port 212. An inspiratory branch 206 extends between a center point 214 of the patient wye 124 and the inspiratory port 204. In addition, the patient wye 124 provides an inspiratory limb connection surface 216 to which an inspiratory tube 220 provided as part of the inspiratory limb 112 is joined to the inspiratory branch 206 of the patient wye 124 at the inspiratory port 204. In some embodiments, the connection between the inspiratory branch 112, tubing 220 and the wye 124 is established by a pressure or friction fitting. Accordingly, the inspiratory limb connection surface 216 may be smooth, or may be textured or contoured to provide a more secure connection. In accordance with still other embodiments of the present invention, the connection surface 216 may include features that are adapted to mate with features provided by or at the end of the tubing 220. Similarly, an expiratory branch 210 extends between the center point 214 of the patient wye 124 and the expiratory port 208. An expiratory limb connection surface 224 is provided for connecting a length of tubing comprising an expiratory tube 228 provided as pail of the expiratory limb 216 of the patient circuit 108 to the expiratory branch 210 of the patient wye 124 at the expiratory port 208. Likewise, a patient branch 218 extends between the center point 214 of the branches of the patient wye 124 and the patient port 212. A patient limb connection surface 232 is provided between the center point 214 of the patient wye 124 and the patient port 212 for connecting a length of tubing comprising a patient tube 236 provided as part of the patient limb 120 of the patient circuit 108 to the patient branch 218 of the patient wye 124 at the patient port 212. The connection surfaces are generally located on an exterior of a wall defining the respective branch of the patient wye 124. In accordance with embodiments of the present invention, the center point 214 of the patient wye 124 may coincide with the point at which the center lines of the inspiratory 206, expiratory 210 and patient 218 branches of the patient wye 124 intersect. In accordance with further embodiments of the present invention, the distance between the center point 214 of the patient wye 124 and any one of the ports 204, 208, 212 is no more than a prescribed distance. In alternative embodiments, the prescribed distance is less than about fifteen millimeters (15 mm), is less than about 20 millimeters (20 mm), or the like. In this manner, a variety of patient circuit components may be used with wye 124. It will be appreciated by those skilled in the art that the prescribed distance may be longer or shorter, as needed to provide a desired fit between wye 124 and the patient circuit components. Additionally, while FIG. 2A depicts the ports 204, 208 and 212 disposed inside the coupled wye connections, in alternative embodiments one or more tube 220, 228 and/or 236 may be inserted into the respective port to couple the tube to wye 124.

The embodiment illustrated in FIG. 2A features a first flow transducer 240a placed in the inspiratory branch 206, between the inspiratory port 204 and the center point 214 of the patient wye 124. In addition, a second flow transducer 240b is disposed in the expiratory branch 210, between the expiratory port 208 and the center point 214 of the patient wye 124. By providing flow transducers 240 within two of the three branches of the patient wye 124, rates of flow through each of the branches of the patient wye 124, and thus through each of the limbs of the patient circuit 108, can be determined, as can be appreciated by one of skill in the art. In particular, flows through the inspiratory and expiratory branches with transducers can be determined directly, while flow through the patient branch can be determined by taking the difference of the flows in the inspiratory and expiratory branches. In addition, the inclusion of flow transducers 240 as part of the patient wye 124 allows flow rates to be monitored, without increasing dead space within the patient circuit 108. In the embodiment illustrated in FIG. 2A, the flow transducer 240a associated with the inspiratory branch 112 is located at a point along the inspiratory branch 112 that is coincident with or adjacent at least a portion of the inspiratory limb connection surface 216, while the flow transducer 240b associated with the expiratory branch 116 is located at a point along the expiratory branch that is coincident with or overlaps at least a portion of the expiratory limb connection surface 224. Because the connection surfaces 216, 224 are a necessary part of a patient wye 124, it can be appreciated that the addition of the flow transducers 240 to the patient wye 124 is accomplished without necessarily adding to the volume of the patient wye 124 and thus without adding to the volume of the patient circuit 108.

FIG. 2B illustrates another embodiment of a patient wye 124 in accordance with embodiments of the present invention. This embodiment features a first flow transducer 240a associated with the inspiratory limb 120 of the patient circuit 108 that is located within the inspiratory branch 206 of the patient wye 124, and a second flow transducer 240b associated with the patient limb 126 of the patient circuit 108 that is located within the patient branch 218 of the patient wye 124. Moreover, in the illustrated embodiment, the first flow transducer 240a is coincident with the inspiratory limb connection surface 216 provided by the patient wye 124 for connecting the inspiratory branch 206 of the patient wye to the tube 220 included in the inspiratory limb 112. Similarly, the second flow transducer 240b is coincident with the patient limb connection surface 232 for connecting the patient branch 218 of the patient wye 124 to the tube 236 associated with the patient limb 120. As can be appreciated by one of skill in the art, by monitoring rates of flow in the inspiratory 112 and patient 120 limbs, flows in those branches, as well as in the expiratory limb 116, can be determined.

FIG. 2C illustrates yet another embodiment of a patient wye 124 in accordance with embodiments of the present invention. In FIG. 2C, a first flow transducer 240a is provided in association with the expiratory limb 116 of the patient circuit 108, while a second flow transducer 240b is associated with the patient limb 120 of the patient circuit 108. The transducer 240a associated with the expiratory limb 116 is located in the expiratory branch 210 of the patient wye 124, and is coincident with the expiratory limb connection surface 224. The transducer 240b associated with the patient limb 120 is located in the patient branch 218 of the patient wye 124, and is further located coincident with the patient limb connection surface 232. As can be appreciated by one of skill in the art, the provision of flow transducers 240 in association with the expiratory limb 116 and patient limb 120 of the patient circuit 108 allows flow rates within those limbs to be determined, as well as flow rates within the inspiratory limb 112.

FIG. 2D illustrates yet another embodiment of the present invention. In FIG. 2D, a flow transducer 240 is associated with each limb of the patient circuit 108, and thus with each branch of the patient wye 124. In particular, a first flow transducer 240a is located in the inspiratory branch 206 of the patient wye 124 for monitoring flows within the inspiratory limb 112. A second flow transducer 240b is located in the expiratory branch 210 of the patient wye 124 for monitoring flows within the expiratory limb 116 of the patient circuit 108. In addition, a third flow transducer 240c is located in the patient branch 218 of the patient wye 124 for monitoring flows in the patient limb 120 of the patient circuit 108. By providing a flow transducer 240 in association within each branch of the patient wye 124, flows in each limb of the patient circuit 108 can be monitored directly, and the signals output by the transducers can be compared in order to verify that all of the transducers 240 are operating properly. Moreover, because the transducers 240 are located opposite at least a portion of the respective connection surfaces 216, 224, 232, the dead space or volume of the patient circuit 108 is not necessarily increased.

In FIG. 2E, a patient wye 124 in accordance with another embodiment of the present invention is depicted. In this embodiment, a single, first flow transducer 240 is provided in the patient branch 218 of the patient wye 124. As shown, the transducer 240 is located at a point along the patient branch 218 that is coincident with or adjacent the connection surface 232 provided by the patient wye 124 for connecting the tubing 236 associated with the patient limb 120 to the patient port 212. The provision of a flow transducer 240 in association with the patient branch 118 allows flows within the patient limb 120 to be monitored. In addition, by providing a flow transducer 240 that is integrated with the patient wye 124, flows through the patient circuit 120 can be monitored without increasing the dead space within the patient circuit 120 as compared to a patient wye without transducers. Accordingly, the transducer 240 is incorporated without increasing the volume of the patient limb 120. In this embodiment, flow transducer 240 may be used to determine inspiratory flow into a patient, and/or expiratory flow coming from the patient.

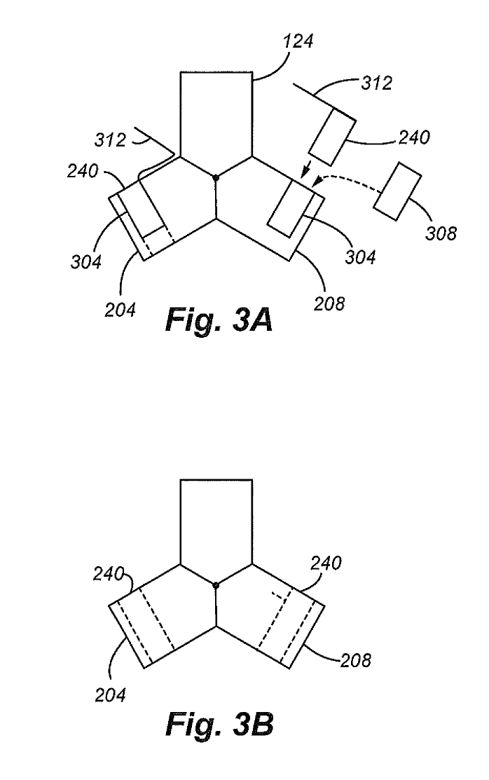

FIGS. 3A and 3B illustrate different ways to integrate one or more flow transducers 240 with a patient wye 124 in accordance with embodiments of the present invention. In particular, in FIG. 3A, a patient wye 124 with a slot or orifice 304 provided in one or more branches for receiving a flow transducer 240 is illustrated. In general, by placing a flow transducer 240 in an orifice 304, the flow transducer 240 is placed in communication with the interior of the patient wye 124, and in particular with the interior of the branch in which the orifice 304 is formed. The orifice 304 is dimensioned so that the transducer 240 can be received in a closely fitting, leak free manner. In addition, by providing an interconnection that is releasable, the flow transducer 240 can be removed if it is not required or for servicing. Where the patient wye 124 is to be operated without a flow transducer 240 in a branch provided with an orifice 304, a cover or cap 308 maybe provided to seal the orifice 304 and the associated branch of the patient circuit 108. As an alternative to a cover 308, the wye 124 can be used without a transducer 240 or a cover 308 to seal a provided orifice 304 where the orifice 304 is entirely covered by the end of a tube provided as part of one of the limbs of the patient circuit 108, when that tube is connected to a connection surface of the patient wye 124 with the open orifice 304.

FIG. 3A also illustrates lead wires 312 from the sensor that can be terminated at the ventilator 104. As can be appreciated by one of skill in the art, the lead wires 312 may provide power to the flow transducers 240, and may carry signals from the transducers 240 to the controller 132 of the ventilator 104. Alternatively, lead wires 312 may be coupled to a separate proximal sensor package or controller, which in turn may be in communication with controller 132 of ventilator 104. Though the example illustrated in FIG. 3A shows transducers 240 associated with the inspiratory 112 and expiratory 116 limbs, it should be appreciated that orifices 304 for receiving flow transducers 240 may be provided in walls defining any one, any two, or all three branches of the patient wye 124.

FIG. 3B depicts an alternate embodiment in which flow transducers 240, shown by dotted lines in the figure, are integrated with the patient wye 124. In such embodiments, the flow transducer 240 may be serviced or removed through an associated port 204, 208, 212, or alternatively may not be user serviceable. Whether placed into the patient wye 124 through an orifice 304 or through a port 204, 208, 212, a transducer 240 may be secured to the patient wye 124 in various ways. For example, a transducer 240 can be secured by a friction fit, a threaded connection, a snap fit, a fastener, an adhesive or any other suitable connection.

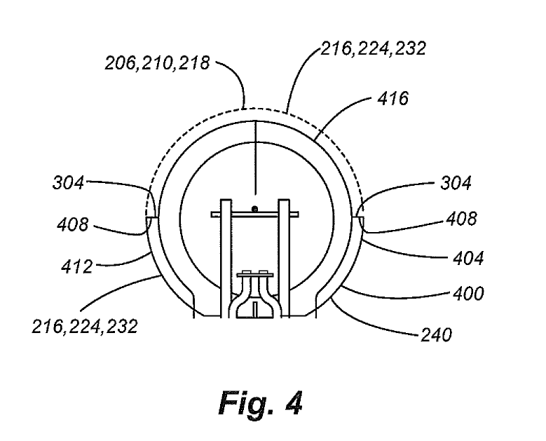

FIG. 4 depicts an example of a flow transducer 240 that can be incorporated into a patient wye 124 in accordance with some embodiments of the present invention. The example illustrated in FIG. 4 depicts a hot wire anemometer 400. As shown, the outer circumference 404 of the anemometer may include steps or shoulders 408 to mate with the edges of an orifice 304 of a branch of the patient wye 124. A raised portion 412 of the outer circumference of the flow transducer 240 with a diameter equal or approximately equal to an outside diameter of a branch of the patient wye may form a portion of the connection surface of the branch of the patient wye 124 with which the flow transducer 240 is associated, while a reduced diameter portion 416 may be sized to fit within the interior of a branch of the patient wye 124. For instance, in the figure, the flow transducer 240 may be associated with the patient branch 218, and thus the raised outer surface 412 of the flow transducer 240 forms a portion of the connection surface 232 of the patient branch 218 of the patient wye 124. In addition, a flow transducer 240 for placement within a patient wye 124 can have other configurations. For example, some or all of the reduced diameter portion 416 can be eliminated, such that the transducer contacts the patient wye 124 only where the edges of the raised portion 412 contact the edges of the orifice 304. In addition, other types of flow transducers can be utilized. Moreover, other types of transducers 240 can be incorporated into a wye 124 in accordance with embodiments of the present invention. For example, the flow transducers 240 may comprise optical sensors incorporating turbines or paddle wheels, orifice flow meters, vortex sensor, or turbine meters. As a further example, a pressure sensor may be used in place of or in conjunction with a flow sensor. In accordance with still other embodiments of the present invention, a composition sensor, for example for detecting the concentration of a particular gas, can be incorporated into a patient wye 124 in addition or as an alternative to a flow transducer 240.

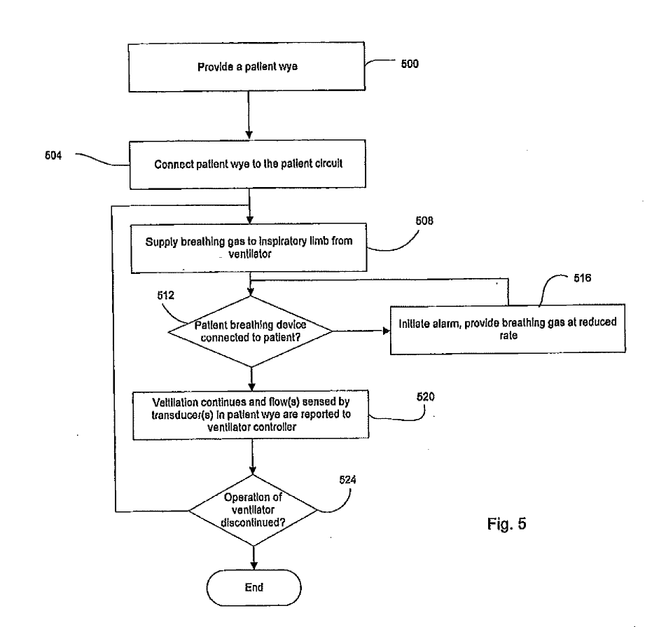

FIG. 5 depicts aspects of a process for providing a patient wye 124 with one or more integrated flow transducers 240 in accordance with embodiments of the present invention. Initially, at step 500, a patient wye 124 is provided. The patient wye 124 includes at least one flow transducer 240 in a branch of the patient wye 124. In accordance with embodiments of the present invention, additional flow transducers 240 can be included. In particular, a flow transducer 240 can be included in any one, any two, or all three branches of the patient wye 124. The one or more flow transducers 240 can be permanently integrated with the patient wye 124, or can be selectively added or removed as desired. In addition to flow transducers 240, other transducers, such as pressure sensors and/or composition sensors, can be added in place of or in addition to flow transducers. Where flow transducers 240 are added via orifices 304 in the conduits forming the branches 206, 210, 218 of the patient wye 124, a blank cover 308 may be used to seal the orifice 304 in place of a transducer 240.

At step 504, the patient wye 124 is connected to the patient circuit 108. As can be appreciated by one of skill in the art, connecting the patient wye 124 to the patient circuit 108 can include connecting tubing 220 extending from the supply port 136 of the ventilator 104 to the patient wye 124 at the inspiratory limb connection surface 216 to complete the inspiratory limb 112 of the patient circuit 108. Connecting the patient wye 124 to the patient circuit 108 can further include connecting tubing 228 to the return port 140 of the ventilator 104 and to the expiratory limb connection surface 224 of the patient wye 124 to complete the expiratory limb 116. Finally, a length of tubing 236 can be connected to a patient breathing apparatus 128 and to a patient limb connection surface 232 to complete the patient limb 120 of the patient circuit 108.

At step 508, breathing gas is supplied to the inspiratory limb 112 from the ventilator 104. While supplying breathing gas, a determination is made as to whether a patient breathing device 128 has been connected to the patient (step 512). If a connection of the breathing device 128 to a patient is not detected, a disconnect alarm can be initiated, and breathing gas can be provided at a reduced rate, to enable detection of a connection by sensing a presence of a flow in the expiratory limb 116 (step 516). After determining that the patient breathing device 128 is connected to a patient, ventilation continues, with the flows sensed by transducers 240 incorporated into the patient wye 124 reported to the ventilator controller 132 (step 520). At step 524, a determination may be made as to whether operation of the ventilator 104 has been discontinued. If operation of the ventilator 104 has not been discontinued, the process may return to step 508. If operation of the ventilator has been discontinued, the process may end.

In accordance with embodiments of the present invention, a calibration of the flow transducers 240 can be performed in various ways. For example and as discussed below, the patient limb 120 can be blocked and flows in monitored limbs can then be determined to make sure that there are no leaks in the patient circuit 108. In addition, calibration can be performed at zero flow. In order to perform multipoint calibration, flow rates detected by different transducers 240 within a patient wye 124 can be compared to one another.

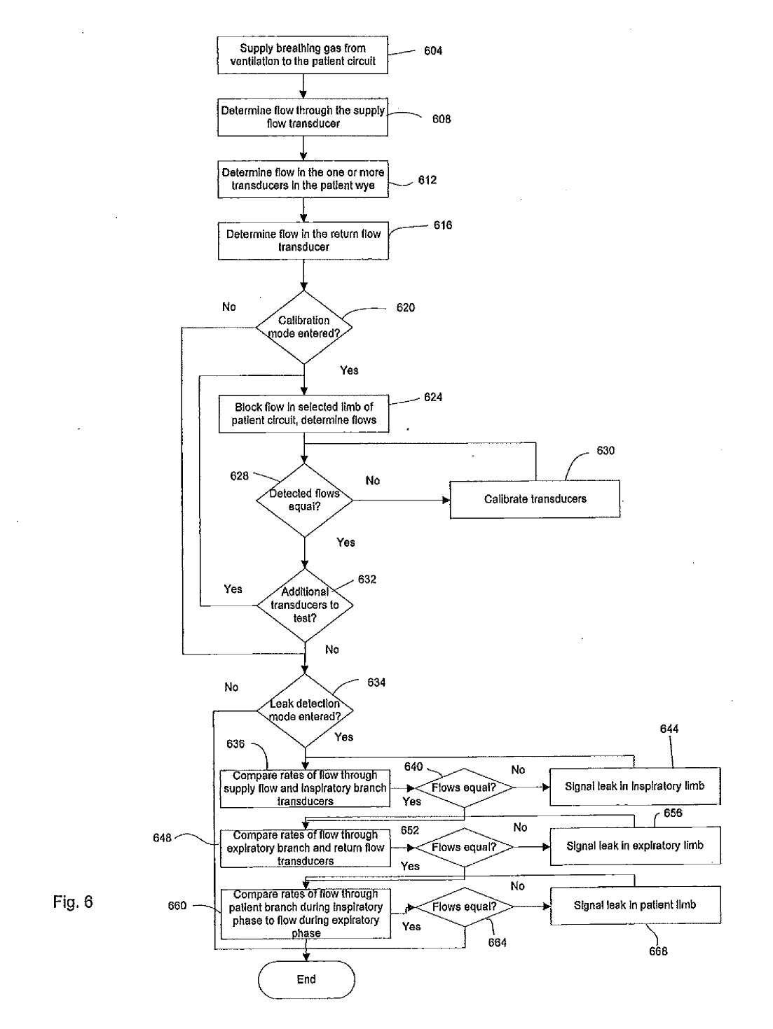

FIG. 6 is a flowchart illustrating aspects of a process for calibrating and detecting leaks in a ventilation system 100 in accordance with embodiments of the present invention. At step 604, breathing gas is supplied from the ventilator 104 to the patient circuit 108. In one embodiment, the flow leaving the ventilator is determined by determining the flow through the supply flow transducer 138 of the ventilator 104 (step 608). In addition, flow in the one or more transducers 240 provided as part of the patient wye 124 is determined (step 612). Flow through the return flow transducer 142 is also determined (step 616) in some embodiments.

At step 620, a determination is made as to whether a calibration mode has been entered. As can be appreciated by one of skill in the art, in a calibration mode, flow transducers included as part of a ventilation system 100, including flow transducers 240 included in or integrated with a patient wye 124, are calibrated to ensure that they provide accurate flow measurements. While performing calibration, in one embodiment one limb of the patient circuit 108 is blocked, and flows detected by the transducers associated with the remaining branches of the patient circuit 108 are compared to one another (step 624). This can be done at multiple flow rates, to provide a multiple point calibration. A determination is then made as to whether the detected flows are equal in the two branches of the patient circuit 108 that have not been blocked (step 628). If the detected flows are not equal, the transducers are calibrated (step 630). Calibrating the transducers can include adjusting individual transducers. For instance, a transducer providing a reading that is determined to be anomalous can be adjusted so that its output matches that of other transducers. As another alternative, values of a plurality of transducers can be adjusted so that they all indicate a common value for a given flow. As yet another alternative, individual transducers believed to be defective can be replaced. After calibrating the transducers, the process may return to step 624 to retest the transducers.

After a determination that flows detected by transducers in the branches of the patient circuit 108 that have not been blocked are equal or differ less than a prescribed calibration amount, a determination is made as to whether additional transducers 240 remain to be tested (step 632). If additional transducers 240 remain to be tested, the process can return to step 624 and another branch of the patient circuit 108 can be blocked, to test the calibration of the transducers associated with one or more other branches. In this way, the transducers of each branch may be tested against the transducers of every other branch in order to perform a full calibration of the ventilator system 100 transducers 138, 142, 240.

Once it is determined that the transducers are calibrated or after a determination that a calibration mode has not been entered, a determination may be made as to whether a leak detection mode has been entered (step 634). In the leak detection mode, flows in the transducers 240 included in one or more of the expiratory 210, inspiratory 216 and patient 218 branches of the patient wye 124 and the supply 138 and return 142 flow transducers provided as part of the ventilator 104 can be compared. For instance, the flow or flows into the branches of the patient wye 124 are compared to the flow or flows out of the patient wye 124 to determine if those flows sum to zero. In accordance with embodiments of the present invention, during an inspiratory phase of the ventilator 104 operation, leak detection can include comparing rates of flow through the supply flow transducer 138 and inspiratory branch 216 transducer 240 (step 636). A determination is then made as to whether the flows are equal (step 640). If the flows are not equal, and in particular if the flow through the transducer 240 in the inspiratory branch 216 is less than the flow through the supply transducer 138, a leak in the inspiratory limb 112 is indicated (step 644). In addition to simply signaling the presence of a leak, the amount or size of the leak can also be indicated. After signaling a leak in the inspiratory limb 112, the process may return to step 636 to verify that remedial steps have been successful at removing the leak.

The expiratory limb flows can also be tested. In particular, during an expiratory phase of the ventilator 104 operation, leak detection can include comparing rates of flow through the expiratory branch 210 transducer 240 and return flow transducer 142 (step 648). If the flow in the expiratory branch 210 transducer 240 is determined to be greater than the flow through the return flow transducer 142 (step 652), a leak in the expiratory limb 116 is indicated (step 656). In addition to simply signaling the presence of a leak, the amount or size of the leak can also be indicated. After signaling a leak in the expiratory branch, the process may return to step 648 to determine whether remedial actions have been successful.

In some embodiments, alternative ways of determining leak in the expiratory circuit are performed. For example, during inspiration, there should be flow through theinspiratory flow transducer and the patient flow transducer. If there is no leak, there should be no flow through the expiratory transducer. However, if there is a leak in the circuit on the exhalation side of the patient wye, then there will be a flow of gas through that leak which will be measurable at the expiratory flow transducer. In some embodiments, the ventilator then checks, or prompts the user to look for leaks in the expiratory limb of the ventilator circuit.

In addition, the patient limb can be tested. In particular, the flow through the transducer 240 in the patient branch 212 of the patient wye 124 during an inspiratory phase is compared to the flow through that same transducer 240 during the expiratory phase immediately following the monitored inspiratory phase (step 660). The flow registered during the inspiratory phase is then compared to the flow registered during the expiratory phase to determine if they are equal (step 664). If the flow during the expiratory period is less than the flow during the inspiratory period, a leak in the patient limb 120 is indicated (step 668). In addition to simply signaling the presence of a leak, the amount or size of the leak can also be indicated. After indicating a leak in the patient limb 120, the process may return to step 660 to determine whether remedial steps have been effective. After determining that flows in and out of the patient limb 120 are equal, the process of leak detection may end.

Although leak detection and calibration processes that determine whether detected flows are equal have been described, absolute equality is not necessary. For example, embodiments of the present invention may signal a leak or indicate that a transducer requires calibration if differences between flows detected by different transducers differ by at least some threshold amount.

In accordance with embodiments of the present invention, the processes of leak detection and/or calibration may be performed by algorithms running on or implemented by the controller 132 provided as part of the ventilator 104. Such algorithms may be implemented as program instructions or code, or as firmware. Moreover, a signal generated to signal a leak in a limb of the patient circuit 108 can be communicated to an operator or user through a human perceptible output provided by the ventilator 104 and operated by the controller 132. Similarly, information regarding the calibration of transducers 138, 142, 240 can be obtained by the controller 132 and output to an operator or user through human perceptible outputs provided by or interconnected to the ventilator 104 and operated in response to signals provided from the controller 132.

The foregoing discussion of the invention has been presented for purposes of illustration and description. Further, the description is not intended to limit the invention to the form disclosed herein. Consequently, variations and modifications commensurate with the above teachings, within the skill or knowledge of the relevant art, are within the scope of the present invention. The embodiments described hereinabove are further intended to explain the best mode presently known of practicing the invention and to enable others skilled in the art to utilize the invention in such or in other embodiments and with various modifications required by the particular application or use of the invention. It is intended that the appended claims be construed to include alternative embodiments to the extent permitted by the prior art.

What is claimed is:

1. A patient wye, comprising:
an inspiratory port;
an expiratory port; and
a patient port, wherein the inspiratory port, the expiratory port and the patient port are in fluid communication with one another;
an inspiratory branch extending between a center point of the patient wye to the inspiratory port;
an expiratory branch extending between the center point of the patient wye and the expiratory port;
a patient branch extending between the center point of the patient wye and the patient port;
a first flow transducer located within a first branch of the patient wye, wherein the first selected branch is a first one of:
a) the inspiratory branch;
b) the expiratory branch; or
c) the patient branch.

2. The patient wye of claim 1, further comprising:
a second flow transducer located within a second branch of the patient wye, wherein the second branch is a second one of:
a) the inspiratory branch;
b) the expiratory branch; or
c) the patient branch.

3. The patient wye of claim 1, wherein the first flow transducer is removably connected to the patient wye.

4. The patient wye of claim 1, further comprising:
an inspiratory limb connection surface, wherein the inspiratory limb connection surface is on an exterior of a wall defining the inspiratory branch;
an expiratory limb connection surface, wherein the expiratory limb connection surface is on an exterior of a wall defining the expiratory branch; and
a patient limb connection surface, wherein the patient limb connection surface is on an exterior of a wall defining the patient branch.

5. The patient wye of claim 4, wherein the first flow transducer is located opposite a first one of said connection surfaces, wherein said first one of said connection surfaces corresponds to said first selected branch.

6. The patient wye of claim 4, further comprising:
a patient breathing apparatus, wherein a length of tubing extends from the patient breathing apparatus to the patient wye, and wherein the length of tubing is connected to the patient wye at the patient limb connection surface.

7. The patient wye of claim 1, further comprising:
a first transducer orifice in a wall of the patient wye defining the first selected branch, wherein the first transducer is placed in fluid communication with an interior of the patient wye through the first transducer orifice.

8. The patient wye of claim 1, wherein the first transducer is one of a flow transducer and a pressure transducer.

9. The patient wye of claim 1, further comprising:
a second transducer located within the first branch of the patient wye,
wherein the first transducer is one of a flow transducer and a pressure transducer, and
wherein the second transducer is a composition sensor.

10. The patient wye of claim 1, wherein the inspiratory port, the expiratory port, the patient port, the inspiratory branch, the expiratory branch, and the patient branch are part of a unitary structure.

11. A method for connecting a patient circuit with a ventilator, the method comprising:
providing a patient wye for connection to the ventilator, wherein the patient wye includes a plurality of branches and a first flow transducer in a first one of the plurality of branches, wherein the first flow transducer is located between a center point of the patient wye and a port of the first one of the plurality of branches; and
sensing a flow in a first one of the plurality of branches with the first flow transducer.

12. The method of claim 11, further comprising:
providing a second transducer in a second one of the plurality of branches, wherein the second flow transducer is located between the center point of the patient wye and a port of the second one of the plurality of branches.

13. The method of claim 11, wherein the providing of the patient wye with the first flow transducer includes placing the flow transducer in an orifice formed in the first one of the plurality of branches.

14. The method of claim 11, further comprising:
connecting an inspiratory port of the patient wye to a supply port of the ventilator;
monitoring a flow of breathing gas supplied by the ventilator at a supply port transducer;
monitoring a flow through the first flow transducer, wherein the first flow transducer is associated with the inspiratory port of the patient wye and is located in an inspiratory branch of the patient wye;
comparing the flow through the supply port transducer to the flow through the first transducer;
in response to determining that the flow through the supply port transducer does not equal the flow through the first transducer, generating a signal indicating the presence of a leak in an inspiratory limb of a patient circuit.

15. The method of claim 11, further comprising:
connecting an expiratory port of the patient wye to a return port of a ventilator;
monitoring a flow of gas through the first flow transducer, wherein the first flow transducer is associated with the expiratory port of the patient wye and is located in an expiratory branch of the patient wye;
monitoring a flow of gas received by the ventilator at a return port transducer;
comparing the flow through the first transducer to the return port transducer;
in response to determining that the flow through the first transducer does not equal the flow through the return port transducer, generating a signal indicating the presence of a leak in an expiratory limb of a patient circuit.

16. The method of claim 11, further comprising:
connecting an inspiratory port of the patient wye to a supply port of a ventilator;
connecting an expiratory port of the patient wye to a return port of a ventilator;
connecting a patient port of the patient wye to a patient breathing apparatus, wherein the patient breathing apparatus is associated with a patient;
monitoring a flow of breathing gas through the first transducer during an inspiratory phase of the ventilator, wherein the first transducer is associated with the patient port of the patient wye and is located in a patient branch of the patient wye;
monitoring a flow through the first flow transducer during a first expiratory phase of the ventilator, wherein the first expiratory phase is the next expiratory phase following the first inspiratory phase of the ventilator;
comparing the flow through the first flow transducer during the first inspiratory phase to the flow through the first flow transducer during the first expiratory phase;
in response to determining that the flow through the first transducer during the first inspiratory phase does not equal the flow through the first transducer during the first expiratory phase, generating a signal indicating the presence of a leak in a patient limb of a patient circuit.

17. The method of claim 11, further comprising:
connecting an inspiratory port of the patient wye to a supply port of a ventilator;
connecting an expiratory port of the patient wye to a return port of a ventilator;
providing a flow of gas to the patient wye;
blocking a first limb of a patient circuit;
sensing gas flows in a second and a third limb of the patient circuit;
comparing the gas flow in the second limb to the gas flow in the third limb;
in response to determining that the gas flows sensed in the second and third limbs are not equal, calibrating a transducer associated with at least one of the second or the third branches.

18. A ventilator circuit, comprising:
a patient wye;
a first transducer integrated into the patient wye;
a patient supply tube interconnected to a patient branch of the patient wye at a first end and to a patient breathing attachment at a second end;
an inspiratory tube interconnected to an inspiratory branch of the patient wye at a first end and adapted to be interconnected to a supply port of a ventilator at a second end; and
an expiratory tube interconnected to an expiratory branch of the patient wye at a first end and adapted to be interconnected to a return port of the ventilator at a second end.

19. The ventilator circuit of claim 18, wherein the first transducer is located between a patient port of the patient wye and a center point of the patient wye.

20. The ventilator circuit of claim 19, wherein the first transducer is located adjacent a patient limb connection surface, and wherein the patient supply tube is interconnected to the patient branch of the patient wye at the patient limb connection surface.

21. The ventilator circuit of claim 18, further comprising a second transducer integrated into the patient wye, wherein the first transducer is located within the inspiratory branch of the patient wye, and wherein the second transducer is located within the expiratory branch of the patient wye.
